(19) United States
(12) Patent Application Publication
(10) Pub. No.: US 2006/0020206 A1
(43) Pub. Date: Jan. 26, 2006

(54) System and method for a virtual interface for ultrasound scanners

(76) Inventors: Luis Serra, Singapore (SG); Chua Beng Choon, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(21) Appl. No.: 11/172,727

(22) Filed: Jul. 1, 2005

Related U.S. Application Data
(60) Provisional application No. 60/585,214, filed on Jul. 1, 2004.
(60) Provisional application No. 60/585,462, filed on Jul. 1, 2004.
(60) Provisional application No. 60/660,858, filed on Mar. 11, 2005.
(60) Provisional application No. 60/660,563, filed on Mar. 9, 2005.

(51) Int. Cl. A61B 8/06

(52) U.S. Cl. 600/447

(57) ABSTRACT

A virtual control system for substantially real-time imaging machines, such as, for example, ultrasound, is presented. In exemplary embodiments of the present invention, a virtual control system comprises a physical interface communicably connected to a scanner/imager, such as, for example, an ultrasound machine. The scanner/imager has, or is communicably connected to, a processor that controls the display of, and user interaction with, a virtual control interface. In operation, a user can interact with the virtual control interface by physically interacting with the physical interface. In exemplary embodiments according to the present invention the physical interface can comprise a handheld tool and a stationary tablet-like device. In exemplary embodiments according to the present invention the control system can further include a 3D tracking device that can track both an ultrasound probe as well as a handheld physical interface tool. In such exemplary embodiments a user can control scan and display functions of the ultrasound machine by moving a handheld tool relative to the stationary tablet, and can perform 3D interactive display and image processing operations on a displayed 3D image by manipulating the handheld tool within a defined 3D space. Alternatively, all control functions, those associated with scan and display control as well as those associated with 3D interactive display and image processing can be mapped to manipulations of the handheld tool in a defined 3D space.

### CROSS REFERENCE TO RELATED APPLICATIONS

This application claims the benefit of the following U.S. Provisional Patent Applications: (i) Ser. No. 60/585,214, entitled “SYSTEM AND METHOD FOR SCANNING AND IMAGING MANAGEMENT WITHIN A 3D SPACE (“SonoDEX”)”, filed on Jul. 1, 2004; (ii) Ser. No. 60/585,462, entitled “SYSTEM AND METHOD FOR A VIRTUAL INTERFACE FOR ULTRASOUND SCANNERS (“Virtual Interface”)”, filed on Jul. 1, 2004; and (iii) Ser. No. 60/660,858, entitled “SONODEX: 3D SPACE MANAGEMENT AND VISUALIZATION OF ULTRASOUND DATA”, filed on Mar. 11, 2005.

The following related United States Patent applications, under common assignment herewith, are also fully incorporated herein by this reference: Ser. No. 10/469,294 (hereinafter “A Display Apparatus”), filed on Aug. 29, 2003; Ser. Nos. 10/725,773 (hereinafter “Zoom Slider”), 10/727,344 (hereinafter “Zoom Context”), and 10/725,772 (hereinafter “3D Matching”), each filed on Dec. 1, 2003; Ser. No. 10/744,869 (hereinafter “UltraSonar”), filed on Dec. 22, 2003, and Ser. No. 60/660,563 entitled “A METHOD FOR CREATING 4D IMAGES USING MULTIPLE 2D IMAGES ACQUIRED IN REAL-TIME (“4D Ultrasound”), filed on Mar. 9, 2005.

### TECHNICAL FIELD

The present invention relates to substantially real-time medical scanning and imaging, and more particularly to a virtual control interface for controlling real-time scanning and display machines in medical contexts.

### BACKGROUND OF THE INVENTION

Effective use of a substantially real-time medical scanner, such as, for example, an ultrasound machine, generally requires a user to control both the position and orientation of a probe as well as the scanning machine itself.

Conventionally, substantially real-time scanning machines, such as for example, ultrasound machines, provide customized mouse and keyboard controls for the scanner, as well as a selection of scanning probes which are attached to the scanner. While scanning a patient, a user (generally a health care clinician; known as a “sonographer” in ultrasound contexts) handles a probe with one hand (for example, the right hand for abdominal scans or the left hand for cardiac scans) and manipulates keyboard and mouse interfaces to the scanning machine with the other. This handiwork must be done by a user as he simultaneously watches a computer monitor or other display where the acquired images are displayed. Given the general complexity of image controls and the close attention to the displayed anatomies that is required for diagnosis and/or intervention, the division of a user's attention in this manner can impede or even degrade his performance of these tasks.

Such image control tasks can include, for example: (i) gain control for an ultrasound signal (conventionally implemented using several slide potentiometers to control the gain at several depths from a probe's tip); (ii) transmit power and overall gain control (conventionally implemented using rotary potentiometers) ; (iii) linear measurements and area/perimeter measurements using elliptical approximation, continuous trace or trace by points (conventionally implemented using a mouse-like track ball for measurements and text positioning); (iv) starting and stopping 3D modes, Doppler mode, panoramic view mode, etc. and controlling each of a mode's particular tools; and (v) Adjusting a probe's scanning depth, and angle of scan (in convex probes), also conventionally implemented using rotary potentiometers.

Additionally, conventional real-time medical scanner interfaces, such as, for example, those to ultrasound machines, are not programmable. In general, once a given functionality is assigned to a particular key, lever or button on a given ultrasound machine, that interface device's functionality cannot be reconfigured. There are sometimes found function keys (such as, for example, F1, F2, etc.) that can be customized by a user, and some buttons can have an integrated light so that they can indicate their active/nonactive status by being on or off. Nonetheless, it is often confusing to have buttons in place that are not active.

Notwithstanding the cumbersomeness of conventional interfaces, state of the art ultrasound machines allow a user to perform numerous image processing functionalities on raw ultrasound data, and these functionalities are capable of being updated, modified, upgraded or reprogrammed. Often such image processing functionalities are specific to a given medical specialty, such as, for example, fetal ultrasound or cardiology. In such cases enhanced ultrasound machines can, for example, automatically calculate cranial size and diameter, head to body ratios, heart and lung size, etc., or can be optimized to display the face of a baby.

Thus, using a set of fixed interface controls which are hard wired to fixed operational and control functions presents a significant problem for real-time scanning interfaces where specialized functionalities and upgrades thereto are becoming more and more common. For example, a designer of an ultrasound scanner has to decide whether to provide a few programmable buttons that can each have many functionalities mapped to them or to use many buttons, where each is dedicated to a specific function. It is noted that the latter choice is good for operators since the needed buttons can be memorized and thus quickly located, but it tends to clutter the keyboard with keys that might never be used.

Additionally, conventional real-time scanning modalities use a series of two-dimensional images to offer insight into what are essentially three-dimensional anatomical structures, such as, for example, fetuses, livers, kidneys, hearts, lungs, etc. Thus, for example, state of the art 3D ultrasound technology converts acquired 2D scan images into 3D volumes, and provides users with 3D interactive display and processing functionality (such as, for example, rotation, translation, segmentation, color look-up tables, zoom, cropping, etc.) to allow users to better depict the actual structures under observation and to operate on the displayed 3D images in a three-dimensional way. It is thus a difficult task to map such 3D display and processing operations to a conventional ultrasound interface, which is simply a keyboard and mouse. It is also a difficult task to ask a user to interact with a standard keyboard-and-mouse type interface for basic image control operations, as described above, and to then use another, perhaps more natural interface, for 3D interaction with a displayed volume. These difficulties will only be further exacerbated as time goes on, as more and more complex 3D interactive functionalities are offered on substantially real-time scanning machines.

What is needed in the art is a control interface for substantially real-time imaging systems that solves the above described problems of the prior art.

### SUMMARY OF THE INVENTION

A virtual control system for real-time imaging machines is presented. In exemplary embodiments according to the present invention, a virtual control system comprises a physical interface communicably connected to a scanner/imager, such as, for example, an ultrasound machine. The scanner/imager has, or is communicably connected to, a processor that controls the display of, and user interaction with, a virtual control interface. In operation, a user can interact with the virtual control interface by physically interacting with the physical interface. In exemplary embodiments according to the present invention the physical interface can comprise a handheld tool and a stationary tablet-like device. In exemplary embodiments according to the present invention the control system can further include a 3D tracking device that can track both an ultrasound probe as well as a handheld physical interface tool. In such exemplary embodiments a user can control scan and display functions of the ultrasound machine by moving a handheld tool relative to the stationary tablet, and can perform 3D interactive display and image processing operations on a displayed 3D image by manipulating the handheld tool within a defined 3D space. Alternatively, all- control functions, those associated with scan and display control as well as those associated with 3D interactive display and image processing can be mapped to manipulations of the handheld tool in a defined 3D space.

### BRIEF DESCRIPTION OF THE DRAWINGS

FIG. 1(a) depicts components of an exemplary ultrasound system controlled via a virtual interface according to an exemplary embodiment of the present invention;

FIG. 1(b) depicts the exemplary system of FIG. 1(a) where a user is operating on the virtual object and the virtual interface has become hidden according to an exemplary embodiment of the present invention;

FIG. 1(c) depicts the exemplary system of FIG. 1(a) where a user has activated the virtual interface by placing an exemplary hand-held tool in the proximity of an interface device according to an exemplary embodiment of the present invention;

FIG. 2 illustrates an exemplary physical interface to a virtual interface, exemplary ultrasound probe, and exemplary ultrasound display positioned near an exemplary patient according to an exemplary embodiment of the present invention;

FIG. 3 is a detailed view of an exemplary user employing an exemplary physical interface to interact with a virtual interface according to an exemplary embodiment of the present invention;

FIG. 4 depicts an exemplary screen view of an ultrasound image and a virtual interface according to an exemplary embodiment of the present invention;

FIG. 4A is a photograph of an actual Technos™ ultrasound machine keyboard (used with permission);

FIG. 4B depicts an exemplary screen view of an ultrasound image and an alternate virtual interface, made to look like the keyboard of FIG. 4A, according to an exemplary embodiment of the present invention;

FIG. 5 depicts an exemplary user scanning a patient and controlling a scanner using a virtual interface according to an exemplary embodiment of the present invention;

FIG. 6 depicts the exemplary user of FIG. 5 interacting with a 3D volume according to an exemplary embodiment of the present invention;

FIG. 7 depicts an exemplary screen shot with an exemplary virtual interface at the bottom and a 3D ultrasound image on top according to an exemplary embodiment of the present invention;

FIG. 8 depicts an exemplary screen shot with the virtual interface of FIG. 7 at the bottom and a magnified view of a portion of the exemplary ultrasound image of FIG. 7 on top according to an exemplary embodiment of the present invention;

FIG. 9 depicts an exemplary screen shot with the virtual interface of FIG. 7 at the bottom and a processed view of a portion of the exemplary ultrasound image of FIG. 7 on top according to an exemplary embodiment of the present invention;

FIG. 10 depicts an exemplary screen shot with the virtual interface of FIG. 7 at the bottom and a magnified view of the exemplary ultrasound image of FIG. 9 on top according to an exemplary embodiment of the present invention;

FIG. 11 depicts an exemplary screen shot with the virtual interface of FIG. 7 at the bottom and the exemplary ultrasound image of FIG. 10 on top shown as a semitransparent solid surface according to an exemplary embodiment of the present invention;

FIG. 12 depicts an exemplary screen shot with a variant virtual interface at the bottom and a fused image on top according to an exemplary embodiment of the present invention; and

FIG. 13 depicts an exemplary screen shot with the virtual interface of FIG. 7 at the bottom and a magnified view of the exemplary ultrasound image of FIG. 12 with a virtual object added on top according to an exemplary embodiment of the present invention.

It is noted that the patent or application file contains at least one drawing executed in color. Copies of this patent or patent application publication with color drawings will be provided by the U.S. Patent Office upon request and payment of the necessary fee.

### DETAILED DESCRIPTION OF THE INVENTION

In exemplary embodiments of the present invention an interface to real-time imaging systems (for example, ultrasound, but in general any scanner that is obtaining images from a body—or object—that need to be seen and interacted in 3D) is provided. An interface according to such exemplary embodiments can, for example, allow a imaging system operator to both work on the imaged body or object in 3D and to control its 2D (and 1D, that is pushing a button) interface, in a ‘seamless’ manner, or a manner that doesn't involve change of tools, that waste time and complicate the procedure.

In exemplary embodiments according to the present invention methods and apparatus for controlling ultrasound scanning machines using a virtual control panel are presented. According to such exemplary embodiments, both standard 2D image control as well as image acquisition and display operations of conventional ultrasound scanners (such as, for example, depth of scan or mode of scan, which are conventionally controlled by a keyboard and mouse) as well as 3D operations on volumetric 3D data (such as, for example, rotating or cropping a 3D volume, zooming into any part of a volume, defining a 3D cutting plane or picking an object within a volume) can be effected. If 3D imaging is not required in a given ultrasound application, an exemplary virtual keyboard according to an exemplary embodiment of the present invention can be used simply to more efficiently control a 2D ultrasound process. This method is significantly advantageous with respect to prior art touch screen interfaces, which are also virtual, in that the interaction space for 2D could be anywhere within easy reach of the clinician, whereas the touch screen requires that the monitor remain within his reach. In exemplary embodiments of the present invention the display is decoupled from the interaction space; if the display is large, so are the interaction gesticulation.

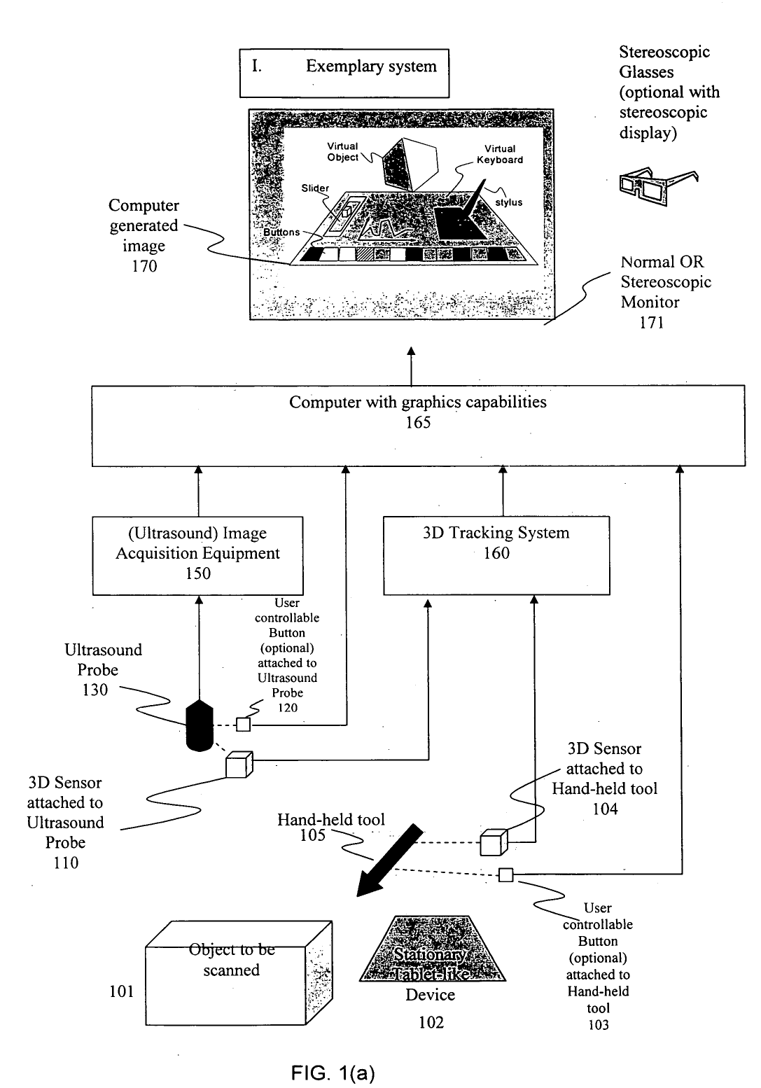

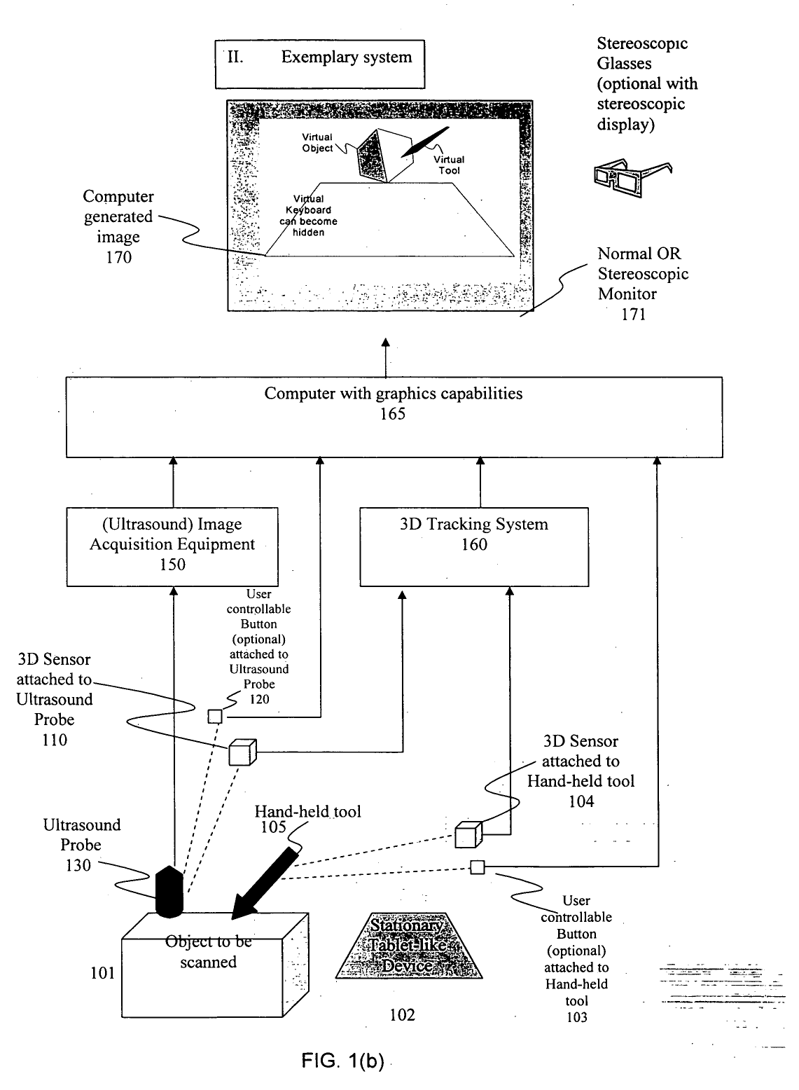

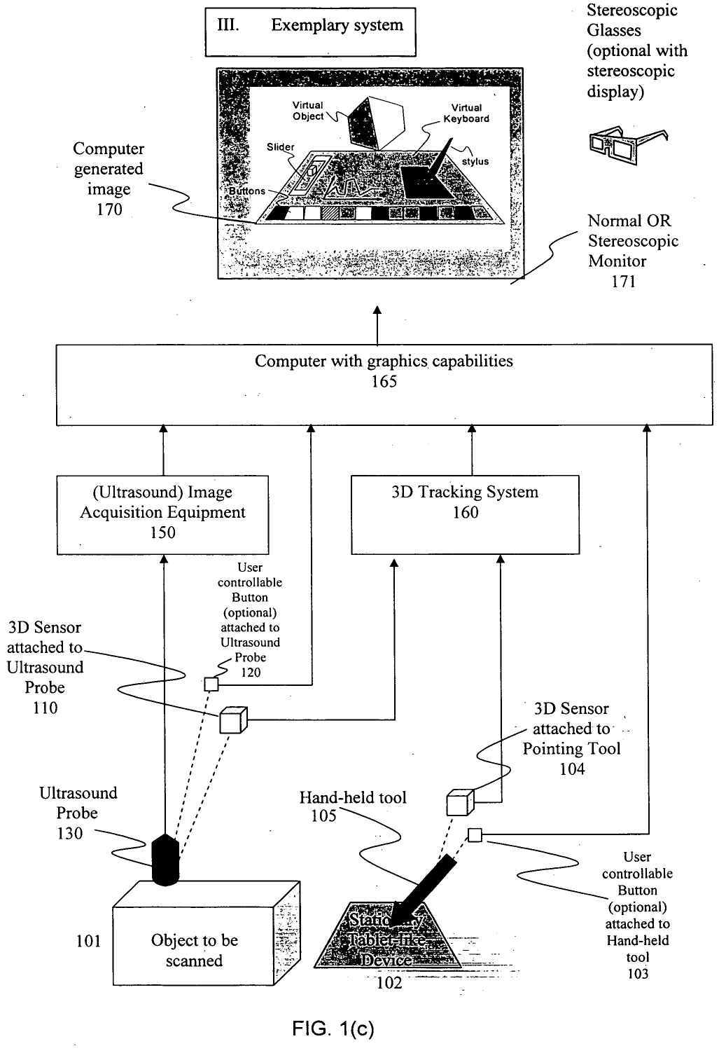

With reference to FIGS. 1(a)-1(c), an exemplary virtually controlled ultrasound system according to an exemplary embodiment of the present invention is depicted. Such an exemplary system contains a computer with graphics capabilities 165, image acquisition equipment 150, and a 3D tracking system. The virtual interface can be, for example, displayed on an ultrasound machine display 171 and interacted with using a variety of physical input devices as may be known. Moreover, a virtual control interface as described herein can have any design as may be desirable, ergonomic or appropriate in given contexts.

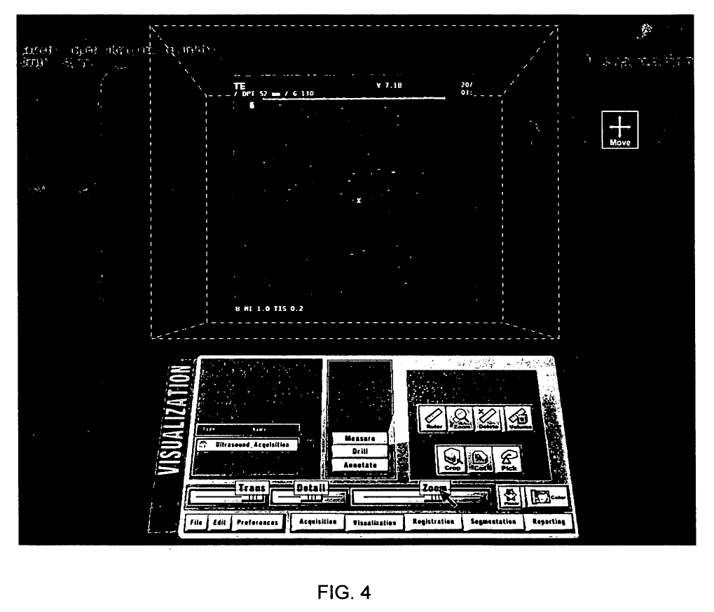

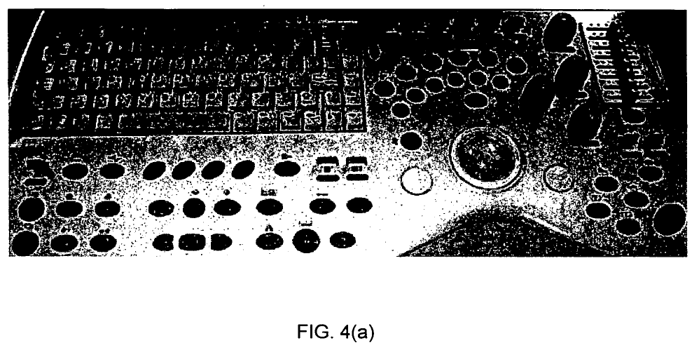

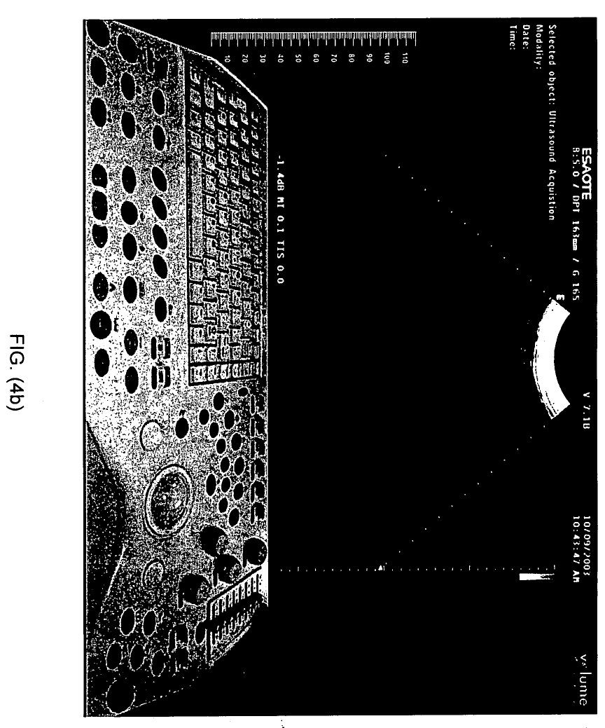

For example, in markets where users are accustomed to the physical keyboard of a conventional ultrasound machine, a virtual interface can appear like a keyboard displayed on the ultrasound machine, i.e., as a “virtual keyboard.” Functions can be, for example, mapped to the virtual keyboard as they are actually mapped to an actual keyboard, and a user, by interacting with the physical interface, can push virtual buttons on the virtual keyboard (and watch them light up or change color on the display to indicate their being virtually “pushed”) precisely as he would have on the actual keyboard. Such a virtual keyboard is shown in FIG. 4B, where the actual Technos keyboard depicted in FIG. 4A is imitated.

For more sophisticated users, a virtual interface can appear more like a standard GUI on a computer, yet can be optimized for the imaging modality being controlled. Such a virtual interface could have, for example, a series of virtual control panels each of which has various function buttons, sliders, display parameters choices or other input/output interfaces. Such a virtual interface would not need to be optimized as to available space and the ergonomics of pushing and reaching real buttons and a physical mouse, but rather could be optimized for ease of identification of control buttons, and for common work flow sequences. For example, for 3D ultrasound machines, the virtual interfaces and palettes provided by Volume Interactions Pte Ltd. of Singapore on its interactive 3D data display system, the Dextroscope™, used in connection with its RadioDexter™ software, could be ported and used as a virtual interface, offering optimized interactive control for a variety of 3D manipulations of data. Such an exemplary virtual interface is depicted in FIG. 4.

As noted, FIG. 1(a) illustrates the components of an exemplary ultrasound system controlled according to an exemplary embodiment of the present invention. With reference thereto, a computer generated image 170 appears on a monitor 171. The monitor can be monoscopic, or stereoscopic, in which case, for example, a user may wear special stereoscopic glasses, or the monitor can be autostereoscopic. The monitor 171 displays an object, being the image of real object 101. Below the virtual object appears a virtual keyboard.

In exemplary embodiments of the present invention a virtual keyboard (as shown in the computer generated image 170), can contain, for example, a slider, various buttons, color look up tables and other graphic interfaces, and can be manipulated by a user using, for example, a hand-held tool 105 which can be grasped much as one grasps a pen. Using such a tool 105, a user can, for example, interact with a surface 102 in a similar way as one interacts with a tablet. This “pen-and-tablet” 105, 102 mechanism can, for example, thus replace a standard physical ultrasound keyboard and mouse (or trackball, etc.). For example, the virtual interface can appear on monitor 171 upon touching the tool 105 to the tablet 102 and then disappear when tool is removed from the surface. A user's position with respect to the tablet 102 can be, for example, tracked using standard tablet tracking systems such as, for example, pressure sensing, or any other known means. The image of the hand-held tool 105 can be, for example, a virtual stylus, as seen in the image 170. Physical motion of the hand-held tool can be, for example, mirrored by virtual motions of the stylus in the image 170. The stylus can be used, for example, to manipulate the virtual keyboard. Thus, in 2D applications, such an exemplary “pen-and-tablet” 105, 102 physical interface can be, for example, all that is required to control an ultrasound scanner.

In alternate exemplary embodiments according to the present invention, a hand-held device 105 can also be tracked by a 3D tracking system 160 as to 3D position and orientation within a defined 3D space. This can be effected, for example, by a hand-held device 105 containing, for example, 3D sensor 104, which can comprise, for example, a radio frequency or optical device which can be “seen” by tracking system 160, or can be effected using other known techniques. Such tracking in 3D space can enable a user to control and/or interact with a displayed 3D ultrasound image obtained from the ultrasound scanner.

For example, a 3D interface can be activated by lifting hand-held tool 105 from the surface of tablet 102 and moving it into a defined 3D space. Such a defined 3D space can be, for example, immediately above tablet 102, or, for example, closer to a patient's body. In the latter case a useful feature according to the present invention is noted. A user, by means of the virtual interface is physically decoupled from the actual scanning machine, and need not be physically proximate to it to control it.

Thus, in exemplary embodiments according to the present invention, a user can control the full functionality of an ultrasound scanner as well as interactively operate in 3D on 3D ultrasound images generated by the ultrasound machine. In such embodiments not only would the virtual stylus be used to interact with the virtual keyboard, but, when the user lifts hand-held tool 105 from the surface of tablet 102 and moves it into a defined 3D space the stylus can operate as a virtual tool, as seen in FIG. 1(b), discussed below.

The virtual stylus could, for example, be active on the virtual object and be used to select portions of the virtual image to operate on using 3D (or 2D) image manipulations and processes in the same manner as a user operates on a 3D data set using a Dextroscope™.

FIG. 1(b) depicts just such an embodiment, where a user has moved the hand-held tool 105 away from the tablet like device 102 and into a defined 3D space. In this space the tool is active with respect to the virtual object, not the virtual keyboard which has become hidden from view in the image 170.

FIG. 1(c) depicts a user once again operating with the virtual keyboard, now visible in the image 170.

Additionally, each of the ultrasound probe 130 and the hand-held tool 105 can have, for example, a user controllable button 120 and 103, respectively, in FIG. 1, which can also be used as an additional physical interface with which to interact with the virtual interface. For example, functions that are frequently required, such as, for example, crop and measure, can be controlled using these buttons in exemplary embodiments.

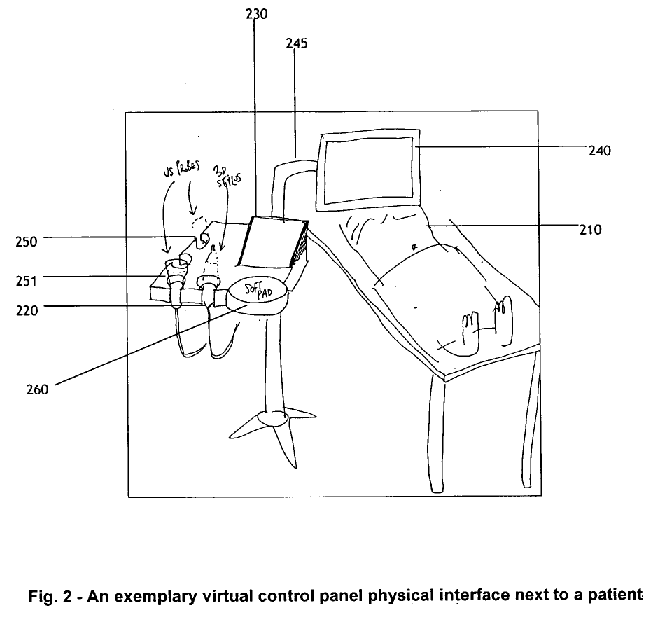

An exemplary physical interface to a virtual control panel and associated apparatus according to an exemplary embodiment of the present invention is next described with reference to FIG. 2. Such physical interface can consist of, for example, a handheld stylus 220 and a tablet 230, here a slanted rigid surface. The slant helps a user to interact, since his hand does not need to reach too far out to touch those buttons farther up. If 3D tracking is used, the tablet becomes just a piece of plastic that serves to rest the pen. If 2D tracking is used then a PDA type pressure sensor can be utilized. The physical interface can be installed on an operator's palette, as shown, which can have several probe holders 250, 251 and a soft pad 260 to provide a comfortable resting place for a user's palm. The physical interface 220, 230 can, for example, be connected to an ultrasound machine with its corresponding monitor 240, the position of which can be held through an arm 245 near a patient 210 and within sight of a clinician. Alternatively, the physical interface can be connected to a dedicated controller or processor, which can in turn be connected to an ultrasound machine.

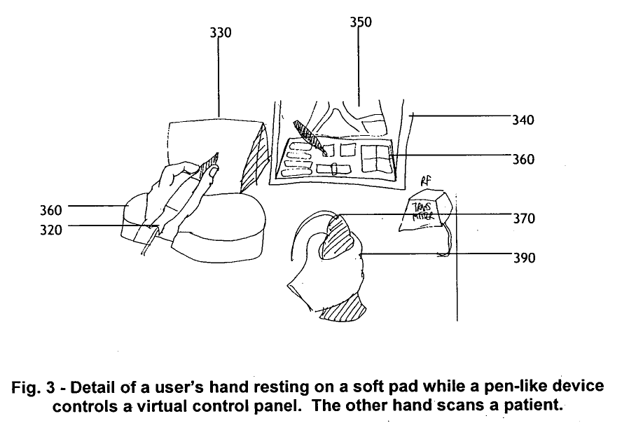

With reference to FIG. 3, in exemplary embodiments of the present invention, in operation a clinician can, for example, rest his palm against soft pad 360, pivoting on the palm of the hand, while holding a tool 320 that, upon contacting tablet 330, can allow a user to control the ultrasound scanner control functions. The user's other hand 390 can, for example, hold an ultrasound probe 370 in the conventional way. A computer monitor 340 can, for example, display both an ultrasound image 350 and a virtual control panel 360 that the user 390 interacts with during the scan.

With reference to FIG. 4, an exemplary screen view is shown. Here, for example, a user sees an ultrasound image 450 on the top of the screen as well as an exemplary virtual keyboard 460 on the bottom of the screen. As noted, the virtual control panel need not be presented in the form of a keyboard, but can take on any desired format as may be convenient, and in general will optimize the virtual controls to ergonomically best suit the physical interface.

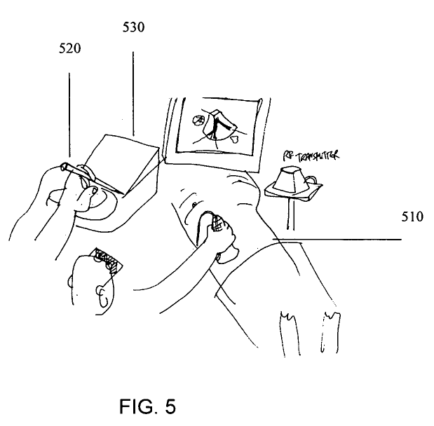

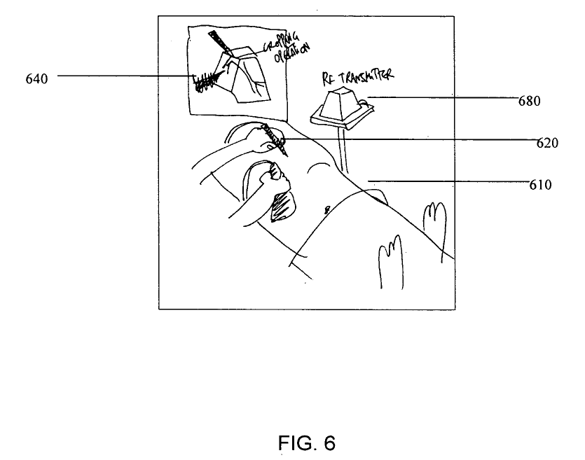

As noted, when combined with a 3D tracking device, a user can both control scan and display parameters as well as operate in 3D space upon a 3D image displayed by the ultrasound machine. This functionality is next illustrated with reference to FIGS. 5 and 6.

In exemplary embodiments according to the present invention, a user can easily shift from controlling scan and display functions to controlling a resulting 3D volume obtained by an ultrasound scanner. Illustrating this functionality, FIG. 5 depicts an exemplary user scanning a patient 510 with his right hand and controlling the scanner with a virtual control panel (not shown) by manipulating a physical interface 520, 530 using his left hand. Similarly, FIG. 6 depicts the same exemplary user shown in FIG. 5 interacting with a displayed 3D ultrasound image 640 by using his left hand to manipulate a tool 620 in a 3D space defined above the patient 610. The tool 620 and the ultrasound probe 670 are each tracked by an exemplary tracking device 680.

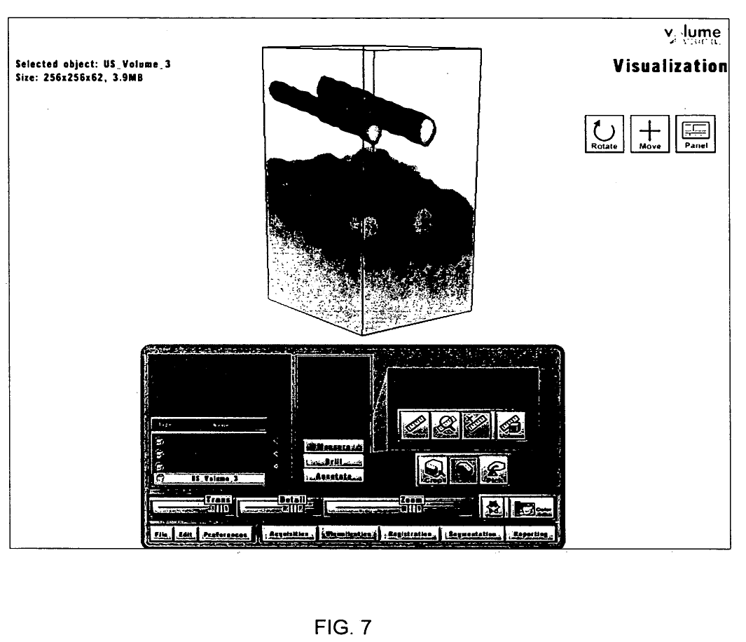

FIGS. 7-13 depict exemplary screen shots according to an exemplary embodiment of the present invention. Each of FIGS. 7-13 depict an exemplary virtual interface according to an exemplary embodiment of the present invention in the bottom portion of the display, and on the top of such virtual interface various 3D ultrasound images are displayed. Each of the 3D ultrasound images depicted in FIGS. 7-13 is the result of a user processing a 3D ultrasound and dataset in some fashion by interacting with the virtual interface depicted in the bottom portion of each figure. These figures are next described in greater detail.

FIG. 7 depicts the ultrasound image of FIG. 4, there shown in 2D, converted into a volume. As can be seen by comparing the ultrasound image of FIG. 4 and the ultrasound image depicted at the top of FIG. 7, the object being scanned is a set of pipes where the scan plane in FIG. 4 is perpendicular to the axes of the pipes. The exemplary image of FIG. 7 has changed the viewpoint and combined a number of planar scans of the pipes into a volume; thus numerous pipes can be seen running from the top left to the bottom right of the depicted ultrasound image. In the depicted exemplary embodiment, this exemplary ultrasound image can be viewed, for example, by choosing one of the volumes via the exemplary virtual interface. As can be seen with reference to the virtual interface, the button “US Volume 3” on the left has been chosen. Also visible are a number of image processing buttons and sliders which can be used to interact with an ultrasound dataset. In the examples of FIGS. 7-13 it is a three-dimensional dataset but it could also be 2D. The various function buttons and sliders available in the exemplary virtual interface will be discussed as is appropriate in connection with the remaining figures.

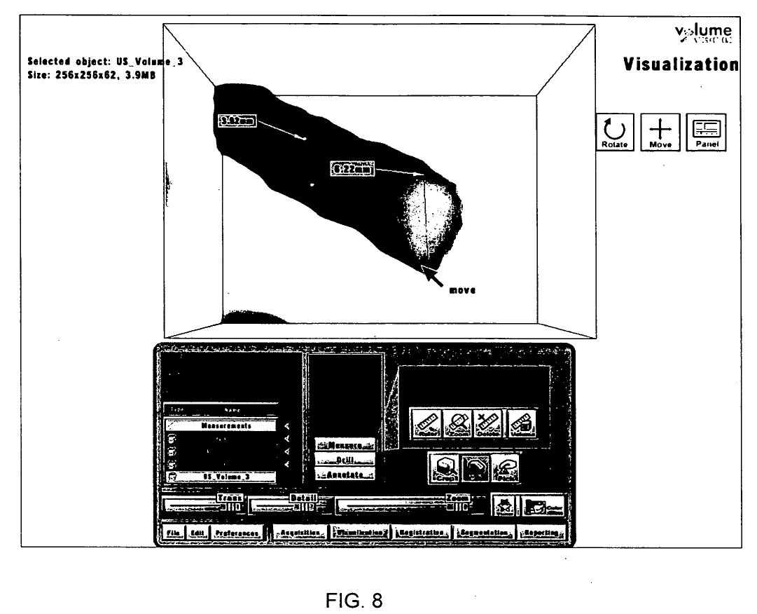

FIG. 8 depicts an exemplary screen view of a user executing a zoom operation on a portion of the dataset visible in FIG. 7. As can be seen with reference to the ultrasound image portion of FIG. 8 (as noted, the virtual interface appears in these exemplary FIGS. 7-13 at the bottom of the display and the actual ultrasound image being displayed appears at the top portion of the display), one of the pipe structures visible in the image of FIG. 7 has been magnified by a user sliding the zoom slider to the right. Such slider is visible in the right-center of the bottom of the virtual interface. As can be seen in the virtual interface of FIG. 8, the zoom slider button has been moved nearly all the way to the right of the available range, indicating that the depicted image is close to maximum zoom. Also visible in the image are a number of points which have been chosen by a user for taking measurements. Finally, also visible in the ultrasound image is an arrow marked “move” by means of which a user can move the depicted object in three dimensions within the display box. As noted above, this type of 3D image processing function can be, for example, implemented by a user lifting the virtual tool above the tablet-like device 105 of FIGS. 1(a)-1(c) and into the defined three-dimensional space where motions of the hand held tool 105 are interpreted as three-dimensional image processing or volume processing commands. In this manner a user can, by moving the hand-held tool 105 through such 3D-defined space, select the object seen in the image of FIG. 8 and move, rotate, take measurements upon, or do a variety of other three-dimensional volumetric processing operations to it, as described in the Zoom Series applications or as are otherwise known in the art.

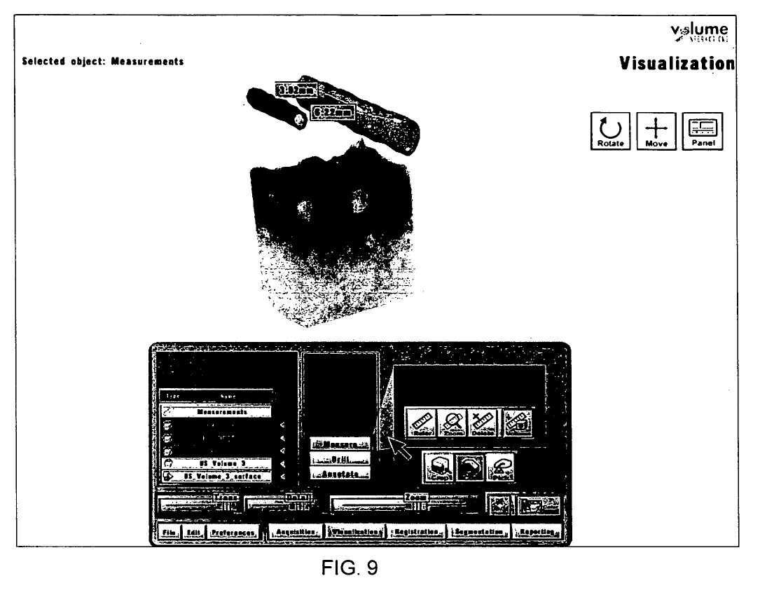

FIG. 9 depicts an exemplary screen shot showing the virtual interface of FIGS. 7-8 at the bottom, and a 3D ultrasound image containing a portion of the dataset depicted in the image of FIG. 7. Moreover, the top right tube of the volume of FIG. 7 has been segmented by a user into a polygonal mesh. Exemplary measurements are also displayed. The front face of the volume has been cropped as well.

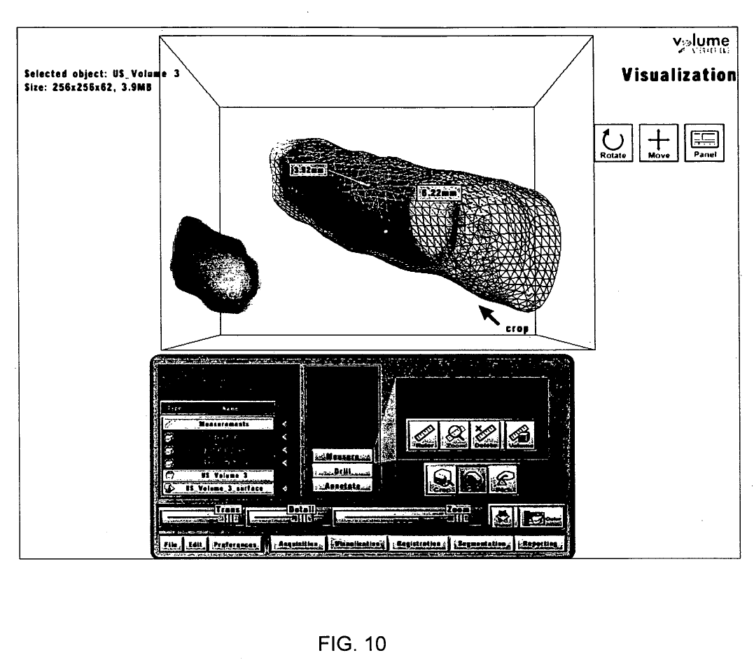

FIG. 9 depicts an exemplary screen shot, where a user has zoomed into the segmented mesh depicted in the image of FIG. 9. Once again, the virtual interface depicted at the bottom of FIG. 10 is identical to the one depicted at the bottom of FIG. 9, except that it indicates a zoom operation has been implemented by a user inasmuch as the zoom slider button is once again nearly all the way to the right of the available range, corresponding to a zoom of the top right tube of the volume displayed in FIG. 9.

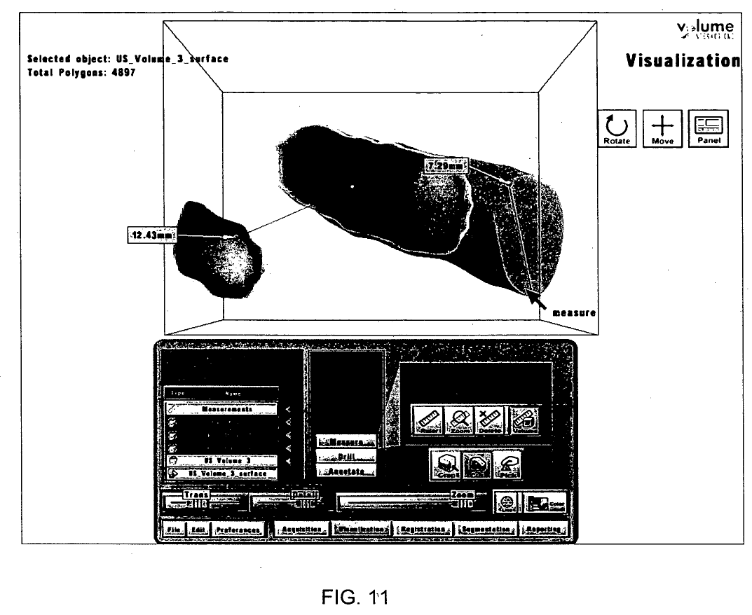

FIG. 11 depicts an exemplary screen shot according to an exemplary embodiment of the present invention. The screen shot depicted in FIG. 11 is the same as that of FIG. 10, but shows the segmented tube as a semitransparent solid surface, as opposed to a segmented mesh. The semitransparent solid surface has been measured across an arc on its cross section in the foreground, and also there has been a measurement between a point on the semitransparent solid surface and one on the next proximate tube in the dataset, shown in the bottom left of the image display box in FIG. 11.

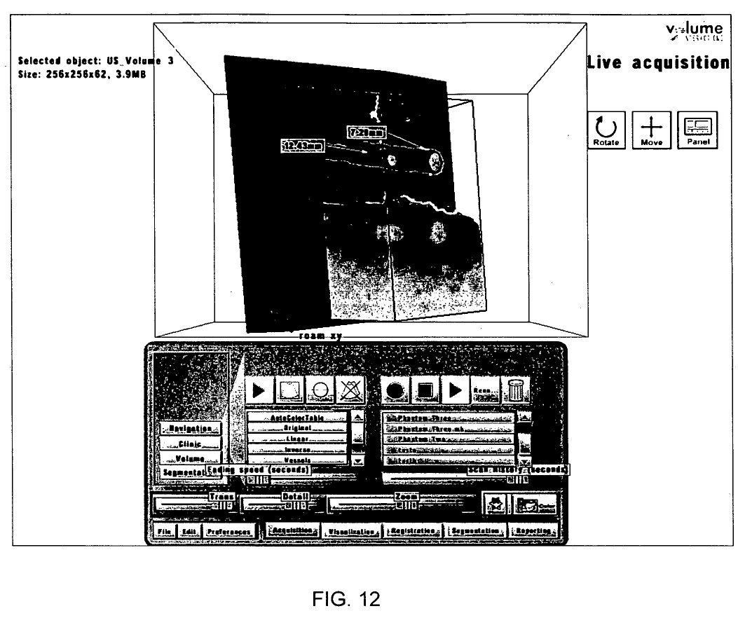

FIG. 12 depicts another exemplary screen shot according to an exemplary embodiment of the present invention. The virtual interface depicted at the bottom of FIG. 12 is somewhat different than that of FIGS. 7-11 because the menu button “Acquisition” (fourth from the left at the bottom of the virtual interface) has been selected as opposed to the “Visualization” button, which is indicated as selected in each of FIGS. 7-11. As can be seen, the virtual interface presents a user with a number of different interactive buttons and slider bars in the “Acquisition” menu. Thus, the image depicted in FIG. 12 is the same as in FIG. 11, with much less zoom (as can be seen by the position of the zoom slider) and a 2D ultrasound slice has been placed in correct 3D positional context and fused with the volume, the segmented tube, and the displayed measurements into one image.

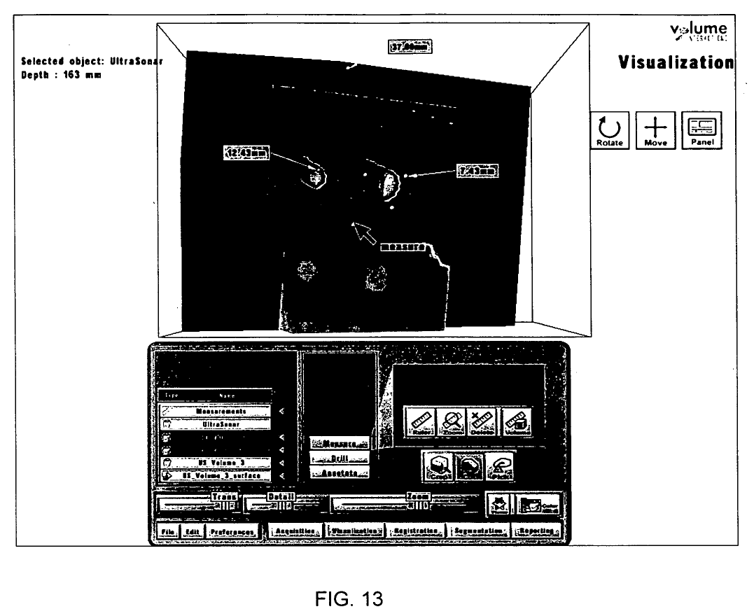

Finally, FIG. 13 depicts an exemplary screen shot according to an exemplary embodiment of the present invention. The depicted screen shot is similar to that of FIG. 12, except that the virtual interface has been shifted back to “Visualization” mode, and its concomitant set of interactive sliders and buttons is therefore depicted. Additionally, the ultrasound image displayed in the display box is the same as depicted in FIG. 12, except that the image has been rotated somewhat, the zoom has been increased slightly, and a user has added an exemplary 3D line (depicted in the display box as a substantially vertical line of length 37.00 mm). This vertical line indicates a virtual planning path which can be inserted into the 3D dataset as a virtual object.

There are various advantages to using a virtual interface according to an exemplary embodiment of the present invention. It allows a user to maintain a uniform line of vision while viewing images derived from an ultrasound or other substantially real-time scan. This contrasts with the conventional viewing of ultrasound images, where a user is required to shift focus from a monitor which displays an image to a keyboard and mouse in order to perform desired control functions. The present invention also solves the problems inherent in certain conventional devices which attempt to partially free a user's hand by mapping certain high-use control functions to an ultrasound probe and the remaining functions to the standard keyboard. Moreover, since a virtual keyboard can be made to appear in similar form as the conventional physical keyboard, users of conventional systems can more easily adapt to the use of a virtual keyboard system.

Another advantage of a virtual control panel and associated physical interface is their ability to be programmed and reprogrammed, as opposed to a fixed control interface which can quickly become outdated or domain restricted. This is of great benefit to the manufacturers, who do not need to build new plastic and electronic interfaces each time new features are added.

While the present invention has been described with reference to certain exemplary embodiments, it will be understood by those skilled in the art that various changes may be made and equivalents may be substituted without departing from the scope of the invention. For example, the disclosed system and method can be used to control, via a virtual interface, any substantially real time medical imaging system. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

What is claimed:

1. A control system for real-time scanning machines, comprising
a physical interface communicably connected to a real-time scanning machine; and
a set of instructions stored on a processor communicably connected to the ultrasound machine, said set of instructions arranged to control the display of and user interaction with a virtual control panel displayed on a scanning machine display;
wherein a user interacts with the virtual control panel by physically interacting with the physical interface.

2. The control system of claim 1, wherein the real-time scanning machine is a medical scanning machine.

3. The control system of claim 2, wherein the real-time scanning machine is an ultrasound machine.

4. The control system of claim 1, wherein said physical interface comprises a stationary rigid tablet and a pen-like tool.

5. The control system of claim 1, further comprising a 3D tracking system, arranged to track both a moveable component of the physical interface as well as a scanning probe.

6. The control system of claim 4, wherein scan and display functions are controlled by manipulating the moveable component relative to a stationary component, and 3D image processing operations on a displayed 3D image are controlled by manipulating the moveable component within a defined 3D space.

7. The control system of claim 4, wherein the defined 3D space is either above a stationary component of the physical interface or above a patient.

8. The virtual control system of claim 1, wherein the physical interface further comprises a soft pad for resting a user's palm.

9. The virtual control system of claim 1, wherein the physical interface further comprises one or more ultrasound probe holders.

10. A method of controlling an ultrasound machine, comprising:
displaying a virtual control panel on a display of the ultrasound machine; and
interacting with said virtual control panel using a physical interface communicably connected to, but physically remote from, the ultrasound machine.

11. The method of claim 10, wherein said physical interface comprises a stationary rigid tablet and a pen-like tool

12. The control system of claim 10, further comprising a 3D tracking system, arranged to track both a moveable component of the physical interface as well as a scanning probe.

13. The control system of claim 4, wherein scan and display functions are controlled by manipulating the moveable component relative to a stationary component, and 3D image processing operations on a displayed 3D image are controlled by manipulating the moveable component within a defined 3D space.

14. The control system of claim 13, wherein the defined 3D space is either above a stationary component of the physical interface or above a patient.

15. The virtual control system of claim 10, wherein the physical interface further comprises a soft pad for resting a user's palm.

16. The virtual control system of claim 12, wherein all control functions are mapped to manipulations of the moveable component in a defined 3D space.

17. A method of controlling a substantially real-time image acquisition and display machine, comprising:
inputting 2D image acquisition and display control commands via a first virtual interface; and
inputting 3D object interaction and manipulational commands via a second virtual interface.

18. The method of claim 17, wherein the first virtual interface is interacted with by means of a 2D physical interface.

19. The method of claim 18, wherein the 2D physical interface is a pen and tablet type device.

20. The method of claim 17, wherein the second virtual interface is interacted with by means of a 3D physical interface.

21. The method of claim 20, wherein the 3D physical interface comprises a 3D tracking system and a tracked hand-held tool.

22. The method of claim 17, wherein the first and second virtual interfaces are the same.

23. The method of claim 17, wherein the first virtual interface is a sub-interface of the second virtual interfaces.

24. The method of claim 17, wherein the substantially real-time image acquisition machine is an ultrasound machine.

25. The method of claim 18, wherein the 2D and 3D physical interfaces are physically remote from the substantially real-time image acquisition machine.
